(19) United States
(12) Patent Application Publication
(10) Pub. No.: US 2017/0258896 A1
(43) Pub. Date: Sep. 14, 2017

(54) ENDOTOXIN NANOVESICLES: HYDROPHILIC GOLD NANODOTS CONTROL LIPOPOLYSACCHARIDE ASSEMBLY FOR MODULATING IMMUNOLOGICAL RESPONSES

(71) Applicant: NATIONAL HEALTH RESEARCH INSTITUTES, Miaoli County (TW)

(72) Inventors: Shu-Yi LIN, Miaoli County (TW); Yueh-Hsia Luo, Miaoli County (TW); Pinpin Lin, Miaoli County (TW)

(21) Appl. No.: 15/069,946

(22) Filed: Mar. 14, 2016

(51) Int. Cl. A61K 39/39; A61K 47/36; A61K 47/02; A61K 9/107

(52) U.S. Cl. CPC A61K 39/39; A61K 9/1075; A61K 47/36; A61K 47/02

(57) ABSTRACT

An endotoxin nanovesicle for enhancing type 1 T helper cell-induced immunological responses is disclosed. The endotoxin nanovesicle comprises: (a) lipopolysaccharide molecules, assembled into a vesicle with a wall surrounding an inner space; and (b) hydrophilic gold nanodots, localized in the wall of the vesicle. Methods of suppressing formation of cubosomes and/or hexosomes in lipopolysaccharide aggregation or assembly, and methods of preparing a lipopolysaccharide adjuvant are also disclosed. Also disclosed are compositions comprising an endotoxin aggregate or an endotoxin nanoversicle and optionally an immunogenic antigen.

### FIELD OF THE INVENTION

The present invention relates to adjuvant, and more specifically to lipopolysaccharide adjuvant.

### BACKGROUND OF THE INVENTION

The endotoxin known as lipopolysaccharide (LPS), which can be obtained from gram-negative bacterial cell walls, is a potent inflammatory activator for inducing immunological responses.¹ By activating the LPS receptor complex, the host immune cells can induce robust proinflammatory cytokines to resist an infection.² Although the excessive production of cytokines may induce systemic inflammatory responses,³ controlling LPS-elicited responses, such as changing the strength or the profile of proinflammatory cytokines, may promote the antigen recognition ability of the host and could potentially be applied as vaccine adjuvants. This potential is due to the fact that LPS forms supramolecular structures consisting of three portions, namely, an O antigen, a core carbohydrate, and a lipid A molecule, and can spontaneously self-assemble to form different types of aggregates.⁴ In general, the LPS aggregate forms easily associate with certain cellular proteins, and these associations can ultimately lead to cell activation that leads, in turn, to the release of cytokines and chemokines.⁵ The various types of LPS aggregates can simply divide into lamellas and non-lamellas, which can exert different strengths in inducing inflammatory responses. Among the non-lamellar aggregates, cubic and hexagonal phases (which are known as cubosomes and hexosomes, hereafter denoted as Q and H, respectively) with particularly strong capacities to induce cytokine expression have been recognized.⁶ At the initial stage, LPS may self-assemble to form lamellar aggregates, dependent on the primary chemical structure of lipid A, which can inactivate cytokine-inducing capacity.⁶⁻⁸ As the LPS aggregates spontaneously change from lamellas to non-lamellar Q and H, a cascade of immunological responses can be gradually evoked, including even the overwhelming production of cytokines, which can lead to various pathophysiological effects such as severe sepsis and other life-threatening consequences.^{6, 9} Since the transition process can modify the intrinsic conformation of the lipid Adomain,¹⁰⁻¹² the cytokine-inducing capability of LPS can be modulated, resulting in the suppression or overactivation of inflammation.¹⁰ The process of transition between lamellas and non-lamellas, however, is random and cannot be well-controlled because the aggregation types are strongly dependent on several parameters, including LPS concentrations, temperatures, and cation categories.¹³⁻¹⁵

To the best of our knowledge, it is still a great challenge to control the self-assembly of LPS molecules with such chemical and structural complexity.

### SUMMARY OF THE INVENTION

In one aspect, the invention relates to an endotoxin aggregate, comprising:
(a) lipopolysaccharide molecules, assembled into a vesicle with a wall surrounding an inner space or a spherical aggregate with a wall surrounding an inner core; and
(b) hydrophilic gold nanodots or gold nanoparticles localized in the wall of the vesicle, or hydrophobic gold nanodots or gold nanoparticles localized in the wall of the of the spherical aggregate.

In one embodiment of the invention, the gold nanodots or gold nanoparticles are embedded within a dendrimer and form a gold nanodot- or a gold nanoparticle-dendrimer complex.

In another embodiment of the invention, the dendrimer has branched amines or branched hydroxyl groups.

In another embodiment of the invention, the dendrimer is a generation-4 dendrimer.

In another embodiment of the invention, the gold nanodot- or the gold nanoparticle-dendrimer complex exhibits a hydrophilic surface polarity.

In another embodiment of the invention, the endotoxin aggregate according to the invention is free of lipopolysaccharide-assembled micelles and/or lipopolysaccharide-assembled lamellas.

In another aspect, the invention relates to an endotoxin nanovesicle, comprising:
(a) lipopolysaccharide molecules, assembled into a vesicle with a wall surrounding an inner space; and
(b) hydrophilic gold nanodots or gold nanoparticles, localized in the wall of the vesicle.

In one embodiment of the invention, the hydrophilic gold nanodots or gold nanoparticles are confined inside of a dendrimer with branched amines.

In another embodiment of the invention, the hydrophilic gold nanodots or gold nanoparticles interact with amine groups of the lipopolysaccharide molecules.

In another embodiment of the invention, the gold nanodots are not alkanethiol-stabilized.

In another embodiment of the invention, the wall of the vesicle has a thickness of about the length of two lipopolysaccharide molecules.

In another embodiment of the invention, the lipopolysaccharide molecules adopt a lipid A-tail-to-lipid A-tail arrangement.

In another embodiment of the invention, the endotoxin aggregate is free of cubosomes and/or hexosomes.

In another embodiment of the invention, the spherical aggregate is a large compound micelle with the inner core filled with reverse micelle.

Further in another aspect, the invention relates to a composition comprising:
(a) the endotoxin nanovesicle or endotoxin aggregate as aforementioned; and
(b) optionally an immunogenic antigen.

Further in another aspect, the invention relates to a method of preparing a lipopolysaccharide adjuvant, comprising:
(a) admixing lipopolysaccharide molecules with hydrophilic gold nanodots or gold nanoparticles; and
(b) allowing the lipopolysaccharide molecules to aggregate and form the endotoxin nanovesicle as aforementioned, and thereby preparing the lipopolysaccharide adjuvant.

Further in another aspect, the invention relates to a method of suppressing formation of cubosomes and/or hexosomes in lipopolysaccharide aggregation or assembly, comprising:
(a) admixing lipopolysaccharide molecules with hydrophilic or hydrophobic gold nanodots or gold nanoparticles; and
(b) allowing the lipopolysaccharide molecules to assemble and form the endotoxin aggregate as aforementioned.

Further in another aspect, the invention relates to a method of suppressing formation of cubosomes and/or hexosomes in lipopolysaccharide aggregation or assembly, comprising:
(a) admixing lipopolysaccharide molecules with hydrophilic gold nanodots or gold nanoparticles; and
(b) allowing the lipopolysaccharide molecules to assemble and form the endotoxin nanovesicle as aforementioned.

Yet in another aspect, the invention relates to a method of enhancing type 1 T helper cell-induced immunological responses in a subject in need thereof, comprising administering to the subject in need thereof an effective amount of the composition as aforementioned, and thereby enhancing the type 1 T helper cell-induced immunological responses in the subject in need thereof.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows the differences in the (a) sizes and (b) surface wettabilities of two kinds of AuNDs. Note that the downward and upward concaves in retention volume ˜20 mL are solvent peaks.

FIG. 2 shows TEM images of the morphologies of LPS aggregates in the presence of AuNDs. The AuNDs-OH/LPS and AuNDs-NH/LPS are shown in the left and right side of Panel (a), respectively. The wall thicknesses and lipid arrangements for individual aggregates were static and are illustrated in panels (b) and (c), respectively. TEM with dark field images are shown in panel (d). The possible fusion of NV_{AuNDs-NH2/LPS} and their size extensions are presented in panel (e, white arrows) and panel (f), respectively.

FIG. 3 shows IL-6 production in endotoxin NV_{AuNDs-NH2/LPS} and LCM_{AuNDs-OH/LPS} treated cells. The levels of IL-6 mRNA and protein were determined by the real-time PCR (a) and ELISA assay (b). The PMA-activated cells treated with endotoxin NV_{AuNDs-NH2/LPS} and LCM_{AuNDs-OH/LPS} for 24 hr (a) or 48 hr (b). *; compared with cell group, p<0.05; #; compared with LPS group, p<0.05.

FIG. 4 shows PCR array comparisons with LPS and NV_{AuNDs-NH2/LPS}. The PMA-activated cells were treated with LPS and NV_{AuNDs-NH2/LPS} for 24 hr. (a) A heat map provides a graphical representation of fold regulation expression data between LPS and NV_{AuNDs-NH2/LPS} overlaid onto the PCR array plate layout (b) The plot displays statistical significance versus fold-change on the y-axis and x-axis, respectively. The P value cutoff value was 0.01, and the boundaries of fold change were 1.5×.

FIG. 5 is a pictorial description of how the endotoxin nanovesicles with dense lipid A units can efficiently modulate immunological responses in human THP-1 macrophages. L (lamellas) and NV_{AuNDs-NH2/LPS} (nanovesicle) presented different LPS-assembled types.

FIG. 6 is a pictroial diagram showing the difference between LPS aggregates in the absence and presence of AuNDs. The arrow 6 indicates that the transformation can stop in the NV in the presence of AuNDs, suppressing the formation of Q/H. The subtypes of the LPS aggregates were not included.

FIG. 7 shows comparisons of zeta potentials between AuNDs and their parent dendrimers.

FIG. 8 shows a comparison of the G₄OH polarities before and after AuNDs entrapment.

FIG. 9a shows AuNDs stabilize LPS aggregative complex formation (AuNDs/LPS). The LPS (1 ug/ml) and AuNDs (1 mg/ml) in were incubated 72 hr and diluted with 4×RPMI medium. The particle sizes by intensity (%) (upper panel) and by number (%) (lower panel) were analyzed by DLS.

FIG. 9b shows LPS-alone presented a time-depended aggregation and disaggregation at room temperature.

FIG. 10 shows the elemental compositions of AuNDS/LPS aggregates were determined using transmission electron microscopy coupled with energy-dispersive X-ray spectroscopy.

FIG. 11 shows size distribution (b) of NV_{AuNDs-NH2/LPS} obtained from TEM images showing in panel (a), counting number about 250.

FIG. 12 shows TEM images of the cubosomes and hexosomes derived from LPS-alone. Note that the observations were performed at high LPS concentrations.

FIG. 13 shows cytotoxicity of AuNDs in THP-1 cells. THP-1 cells were activated by PMA for 3 days, and cells were then cultured with serum-free medium for an additional 24 hr after removal of the PMA. Cytotoxicity was determined at 24 hr by MTT assay. The AuNDs failed to induce cytotoxicity in PMA-activated THP-1 cells.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the terms “nanocluster” and “nanodots” are interchangeable. The term “nanodots” refers to particles with diameters smaller than 2 nm or composed of less than 100 atoms.

The term “gold nanoparticles” refers to spherical gold particles with diameters ranging from larger than 2 nm to 100 nm.

Dendrimers are repetitively branched molecules. A dendrimer is typically symmetric around the core, and often adopts a spherical three-dimensional morphology. Dendrimers are also classified by generation, which refers to the number of repeated branching cycles that are performed during its synthesis. For example if a dendrimer is made by convergent synthesis, and the branching reactions are performed onto the core molecule three times, the resulting dendrimer is considered a third generation dendrimer. Each successive generation results in a dendrimer roughly twice the molecular weight of the previous generation. The first, the second, and the third generation dendrimers are designated as generation-1 (G-1), generation-2 (G-2) and generation-3 (G-3) dendrimers, respectively. Dendrimer-entrapped gold nanoparticles are well-known in the art.

The terms “confined”, “trapped”. “caged”, and “entrapped” are all interchangeable.

End-group of dendrimer is also generally referred to as the “terminal group” or the “surface group” of the dendrimer. Dendrimers having amine end-groups are termed “amino-terminated dendrimers. The terms “dendrimer with branched amines” and “amine-terminated dendrimer” are interchangeable.

The term “treating” or “treatment” refers to administration of an effective amount of a therapeutic agent to a subject in need thereof with the purpose of cure, alleviate, relieve, remedy, ameliorate, or prevent the disease, the symptoms of it, or the predisposition towards it. Such a subject can be identified by a health care professional based on results from any suitable diagnostic method.

“An effective amount” refers to the amount of an active agent that is required to confer a therapeutic effect on the treated subject. Effective doses will vary, as recognized by those skilled in the art, depending on routes of administration, excipient usage, and the possibility of co-usage with other therapeutic treatment.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

HED may vary, depending on other factors such as the route of administration.

Abbreviations: CCR2, CC chemokine receptor 2; CCL2, CC chemokine ligand 2: CCR5, CC chemokine receptor 5; TLC, thin layer chromatography.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Methods

Materials.

The G₄NH₂ dendrimer, G₄OH dendrimer, HAuCl₄, and LPS (Escherichia coli 0111:B4) were obtained from Sigma. Inc. (San Diego, Calif., USA); MWCO membrane filter was purchased from Millipore (PES membrane); WST-8 was obtained from Dojindo Laboratories (Kumamoto, Japan). AuNDs were synthesized by following a previously published procedure.¹ Briefly, 150 mM of HAuCl₄ (200 μL) was added to 20 mL of deionized water containing G₄NH₂. This solution was incubated at 4° C. overnight, and then irradiated using microwaves at 120° C. for 30 min (CEM, Discover LabMate System). The precipitations and AuNDs after reduction were filtered through the MWCO membrane filter (3 KDa). Extra AuCl₄⁻ was removed using anionic exchange chromatography (Merck, FRACTOGEL® EMD TMAE Hicap).

Surface wettabilities of AuNDs.

The wetting/dewetting curves were performed using a TA instruments relative humidity (RH) perfusion microcalorimeter equipped with a TAM III thermostat. The RH was kept constant at 10% until reaching a stable heat flow (signal ranging within −1 to +1 μW). The RH was then maintained in a well-controlled range from 10% to 90% (black line) for all measurements. The TAM II was thermostatically maintained at room temperature (25° C.±1° C.) to calibrate empty stainless ampoules (4 mL ampoule) before starting the experiments. All measurements were carried out on 30-50 mg samples.

Surface Polarity of AuNDs-OH.

All fluorescence spectra were measured in the presence of pyrene (8×10⁻⁸ M) in water. Variations in the intensity ratio of the pyrene fluorescence peaks were observed based on various AuNDs-OH and G₄OH concentrations. The y-axis is the ratio of I₁/I₃; I₁ and I₃ are the intensities at 372 nm and 383 nm, respectively.

Dynamic Light Scattering Analysis and Zeta Potential Measurements of AuNDs.

Size was determined using the (Malvern Zetasizer, Nano-ZS) dynamic light scattering instrument with an argon laser (λ=633 nm, detector angle=173°, and a typical sample volume=100 μL). An aliquot (1 μL, 100 mg/mL) of two types of AuNDs was mixed with LPS (1 μL, 100 μg/mL) in the RPMI-1640 medium (sera-free) at room temperature for size measurement. After 72 h of incubation, the mixtures of AuNDs and LPS were serially diluted with equal volumes of the RPMI medium to measure the AuNDs/LPS complex size. Each sample was measured in triplicate for statistical analysis. Zeta potential was measured using a zetasizer nano system (Zetasizer Nano ZS. Malvern Instruments, Worcestershire, UK). The test sample was mixed with G₄NH₂ (6 μL), AuNDs-NH₂ (25 μL), and AuNDs-OH (25 μL) in deionized water (800 μL). All measurements were conducted at room temperature. Each parameter was measured in triplicate to fit the statistical analysis. See Luo et al (“Endotoxin nanovesicles: hydrophilic gold nanodots control lipopolysaccharide assembly for modulating immunological responses” American Chemical Society, Nano Lett. 2015, 15, 6446-6453), which is herein incorporated by reference in its entirety.

Sizes of AuNDs Determined Using Gel Permeation Chromatography (GPC).

The sizes of AuNDs were analyzed by GPC with using a solution (pH 3) of 0.2 M NaNO₃ and 0.5 M CH₃COOH; Column type: ShodexR-SB-802.5 HQ, elution speed: 0.5 ml/min; separation temperature: 40° C.

Transmission Electron Microscopy.

The mixtures of LPS and individual AuNDs, AuNDs-NH₂, and AuNDs-OH were prepared in RPMI 1640 medium. Samples were mounted on a 400-mesh Cu grid and carbon support and stained with 2% uranyl acetate solution. Excess staining reagent was removed using a filter paper, and the grid was dried prior to transmission electron microscopy measurements (Hitachi H-7650, Japan) at 100 kV and field emission gun transmission electron microscope at 200 keV (JEOL, JEM-2100F, Japan).

Cell Culture.

In the present study, we used a THP-1 cell line, which is a human acute monocytic leukemia cell line. The THP-1 cells were grown as suspension cultures, which can be differentiated into macrophage-like cells by using phorbol 12-myristate 13-acetate (PMA 100 nM, Sigma-Aldrich). The cells were cultured in the RPMI 1640 medium with 2 mM L-glutamine, 1.5 g/L sodium bicarbonate, 4.5 g/L glucose, 10 mM HEPS, 1.0 mM sodium pyruvate, 0.05 mM 2-mercaptoethanol, and 10% fetal bovine serum.

WST-8 Cell Viability Assay.

Cytotoxicity was determined using the microtiter WST-8 assay.² It is a sensitive colorimetric assay for determining cell viability in cell proliferation and cytotoxicity assays. WST-8 is a highly water-soluble tetrazolium salt, which can be reduced by dehydrogenase activities in cells to obtain a yellow-color formazan dye in the culture media. The amount of the formazan dye produced by the activities of dehydrogenases in cells is directly proportional to the number of living cells. An appropriate number of cells (5×10⁴/well) were plated in 96-well microtiter plates and then treated with 100 nM PMA for 3 d. The fresh sera-free medium was replaced for 1 d and the cells were then treated with various concentrations of AuNDs for 24 h. The WST-8 reagent solution (10 μL) was added to each well of the 96-well microplate containing 100 mL of cells in the culture medium, and the plate was then incubated for 2 h at 37° C. Absorbance was measured at 450 nm by using a microplate reader. The relative viability was expressed as a percentage of the nontreated control. The AuNDs-only group was examined to eliminate the interference of WST-8 measurement (data not shown).

Quantitative Real-Time Reverse Transcription Polymerase Chain Reaction Assays.

PMA-activated cells (2×10⁶ cells; 6-cm culture dish) were treated with AuNDs for 24 h and RNA was then extracted using an RNAZOL® RT kit (Life Technologies, Rockville, Md. USA). The purified RNA was stored at −80° C., and cDNA was synthesized using total RNA (3 μg). Quantitative PCR was used to measure IL-6, and the assays were performed using the assay-on-demand gene expression assay mix (Applied Biosystems. Foster City, Calif., USA). Quantitative PCR to measure IL-6 and GAPDH was performed using TAQMAN® universal PCR master mix (Applied Biosystems, Foster City, Calif., USA). The reaction mixture was prepared by mixing aliquots of cDNA, 0.5 μL of the assay-on-demand gene expression assay mix, and 5 μL of TAQMAN® universal PCR master mix (Applied Biosystems, Foster City, Calif. USA) in a final volume of 10 μL. The reaction mixture was analyzed using an ABI PRISM 7900 sequence detector system (Applied Biosystems, Foster City, Calif., USA) with the following PCR program: 95° C. for 10 min followed by 40 cycles of 60° C. for 1 min and 95° C. for 15 s. Quantitative values were obtained from the threshold cycle (Ct) number. The relative mRNA levels of the target genes were derived using the equation 2^{-ΔCt}, where ΔCt=Ct_{target gene}−Ct_{GAPDH}. Data are presented as the fold relative to the control value.³

IL-6 Enzyme-Linked Immunosorbant Assay.

PMA-activated THP-1 (3×10⁵ cells/well; 24-well plate) cells were treated with LPS, AuNDs, or LPS+AuNDs for 2 d, IL-6 concentration in the medium was determined using a human IL-6 ELISA kit (R&D Systems, Inc.) according to the manufacturer's instructions.

Human Cytokine and Chemokine PCR Array.

PMA-activated THP-1 cells were seeded in a 6 cm dish (1×10⁵) and then treated with LPS and LPS+AuNDs-NH₂ for 24 h. RNA was extracted using an RNAZOL® RT kit. The purified RNA was stored at −80° C., and cDNA was synthesized using total RNA. Quantitative PCR was used to measure cytokine and chemokine gene expressions, and the assays were performed using the cytokine and chemokine RT2 profiler PCR array (QIAGEN, GmbH, Germany). The data analysis was performed by QIAGEN Technologies.

Luminex Human Cytokine Enzyme-Linked Immunosorbent Assay.

PMA-activated THP-1 cells (3×10⁵ cells/well; 24-well plate) were treated with LPS, AuNDs-NH₂, and AuNDs-NH₂/LPS for 2 d. Human cytokines were detected and measured using Luminex, the BIO-PLEX® multiplex system (Bio-Rad, BIO-PLEX® Pro Human Cytokine 27-Plex Panel), according to the manufacturer's instructions. The Luminex ELISA assay contains IL-1b, IL-1ra, IL-2, IL-4, IL-5, IL-6, IL-7, IL-8, IL-9, IL-10, IL-12 (P70), IL-13, IL-15, IL-17A, basic FGF, eotaxin, G-CSF, GM-CSF, IFN-r, IP-10, MCP-1. MIP-α, MIP-β, PDGF-BB, RANTES, TNF-α, and VEGF.

Statistical Analysis.

The statistical analysis was conducted using Prism 4.0 software (GraphPad Software, San Diego, Calif., USA). The cytokine and chemokine arrays were analyzed for significant differences by unpaired t test. Differences were considered statistically significant at *p<0.05. **p<0.01, and ***p<0.001.

### Results

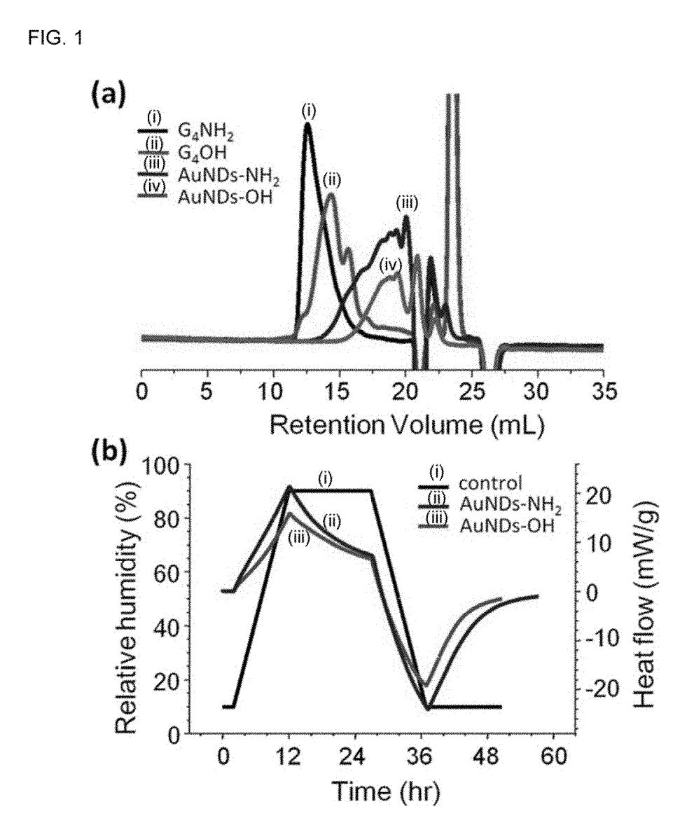

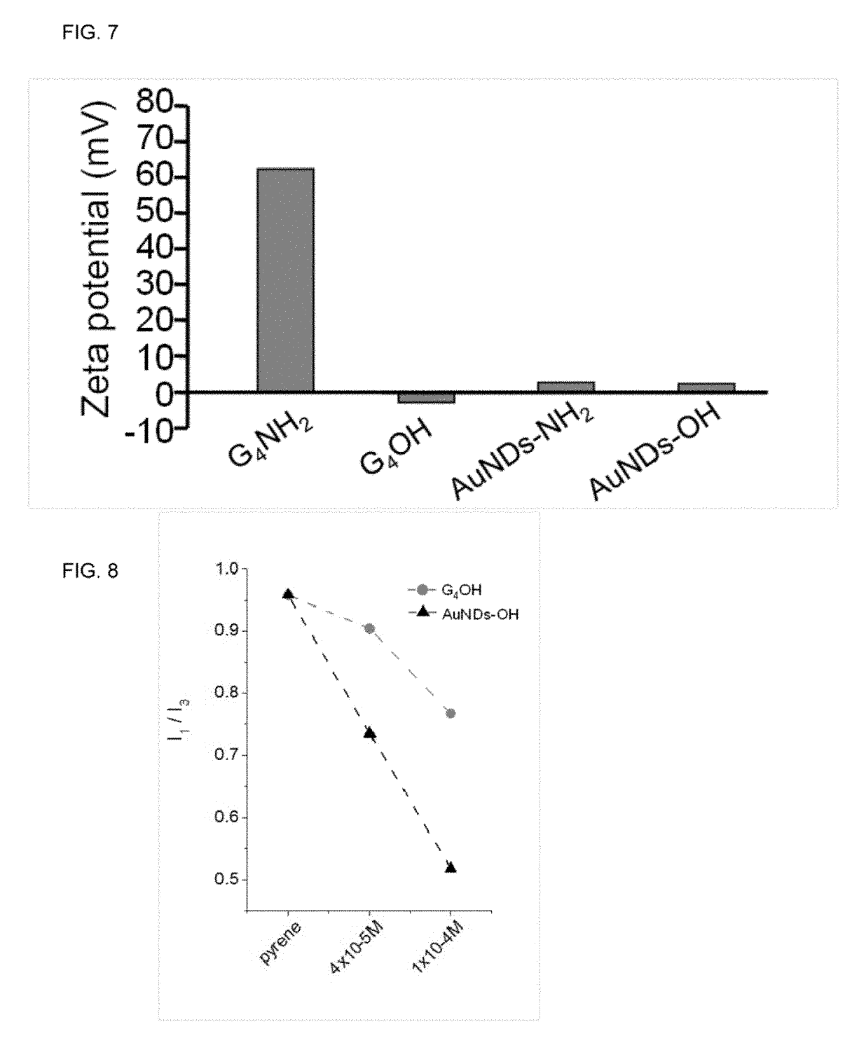

In the present study, we designed a simple strategy based on manipulating the surface hydrophilicity of gold nanodots (AuNDs) to control supramolecular LPS assembly as a means of producing endotoxin NV. Since theoretical studies have shown that the incorporation of gold nanoparticles in the vesicle formation has a size-selective limitation,³² we adopted AuNDs as a candidate to fit this criterion. In addition, alkane thiol-stabilized AuNDs can cause attractive interactions between the AuNDs themselves, and decrease the co-assembly of nanoparticles and amphiphilic molecules.²⁶ Therefore, we directly prepared hydrophilic and hydrophobic AuNDs (denoted as AuNDs-NH₂ vs. AuNDs-OH), by using two outfacing groups of fourth-generation (G₄) dendrimers with branched amine (G₄NH₂) and hydroxyl groups (G₄OH), respectively. Various physicochemical properties of both types of AuNDs, including their sizes, surface charges, and surface wettabilities, were examined, and the related data are shown in FIG. 1. First, through the separation of size-exclusive column (FIG. 1a), both the AuNDs-NH₂ and AuNDs-OH showed lower retention volumes compared to those of their original dendrimers (i.e., G₄NH₂ and G₄OH, data for which are shown in the black line and the red line, respectively, as control sets). These differences indicated that the dimensions of the AuNDs-NH₂ and the AuNDs-OH are smaller than those of their parent dendrimers. These results are consistent with our previous report,³³ in which it was shown that structural contraction can be initiated by a specific interaction between the dendrimer backbone and AuNDs. Secondly, the zeta potentials of two types of dendrimers with or without embedding AuNDs were found to be significantly different, as shown in FIG. 7. Unlike the G₄NH₂ with highly positive charges (˜64 mV), the surface charge of AuNDs-NH₂ is dramatically dropped to ˜3 mV, a value which is almost equal to those of G₄OH and AuNDs-OH. Thirdly, the surface wettabilities of AuNDs-NH₂ and AuNDs-OH were examined via isothermal microcalorimetry, which is a sensitive tool for monitoring the heat changes associated with the wetting/de-wetting process.³⁴ FIG. 1b shows the thermograms with wetting and dewetting curves from the AuNDs-NH₂ (blue line) and AuNDs-OH (green line), respectively, which were measured by the well-controlled humidity ranging from 10% to 90% (black line). Comparatively, the moisture adsorption and desorption values of the AuNDs-NH₂ were higher than those of the AuNDs-OH. These results indicated that the surface hydrophilicity of AuNDs-NH₂ is higher than that of AuNDs-OH. Meanwhile, we used a well-known polarity probe (i.e., pyrene)³³ to clarify whether or not the surface polarity of AuNDs-OH is hydrophobic. The original G₄NH₂ with the charge-to-charge repulsion forces can extend the branch-to-branch space to reverse the original polarity from hydrophobic to hydrophilic. However, the branch-to-branch space of G₄OH remains small, causing the hydrophobic status of G₄OH to be retained.³⁶ As shown in FIG. 8, the ratio of the fluorescence intensities of pyrene at approximately 370 nm (I₁) and 380 nm (I₂) is very similar to that of the original G₄OH. This result indicates that the surface polarity of AuNDs-OH still retains the original hydrophobicity from the dendrimers. Taken together, the results indicate that the surface polarities of AuNDs-NH₂ and AuNDs-OH are prone to hydrophilic and hydrophobic, respectively.

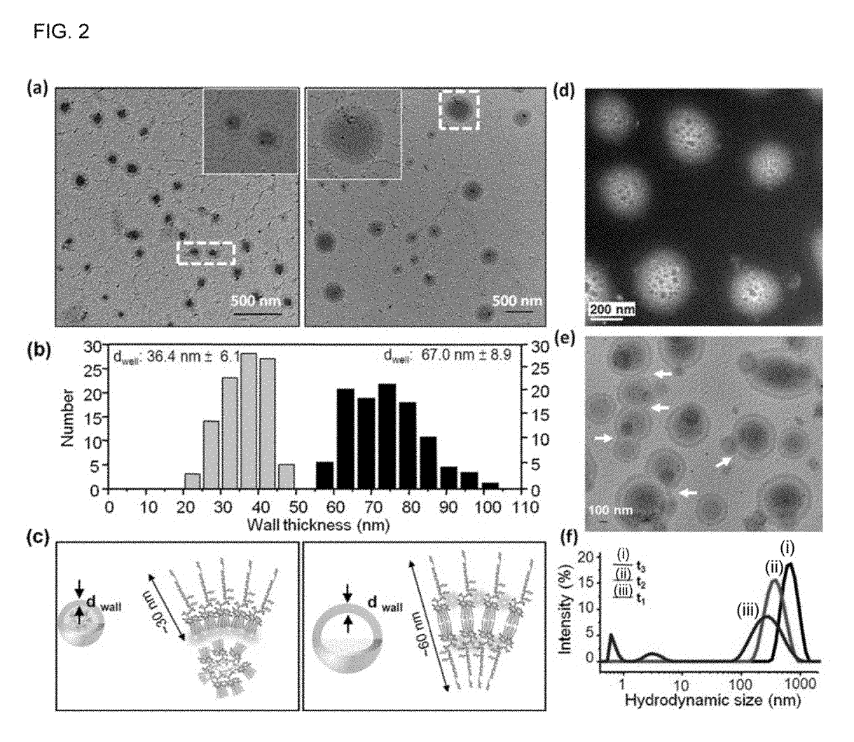

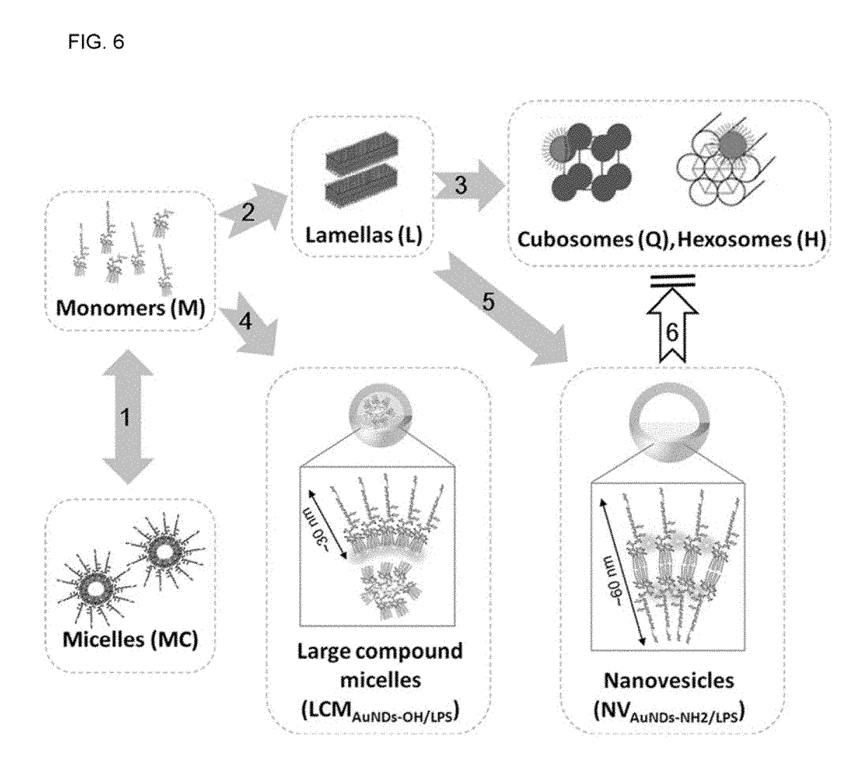

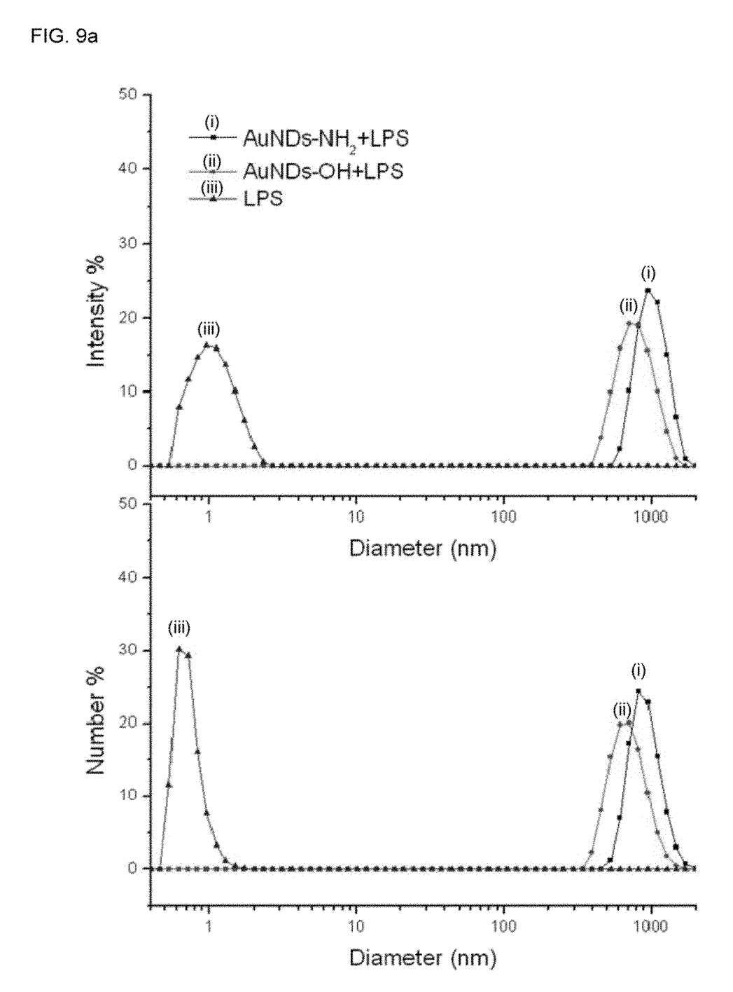

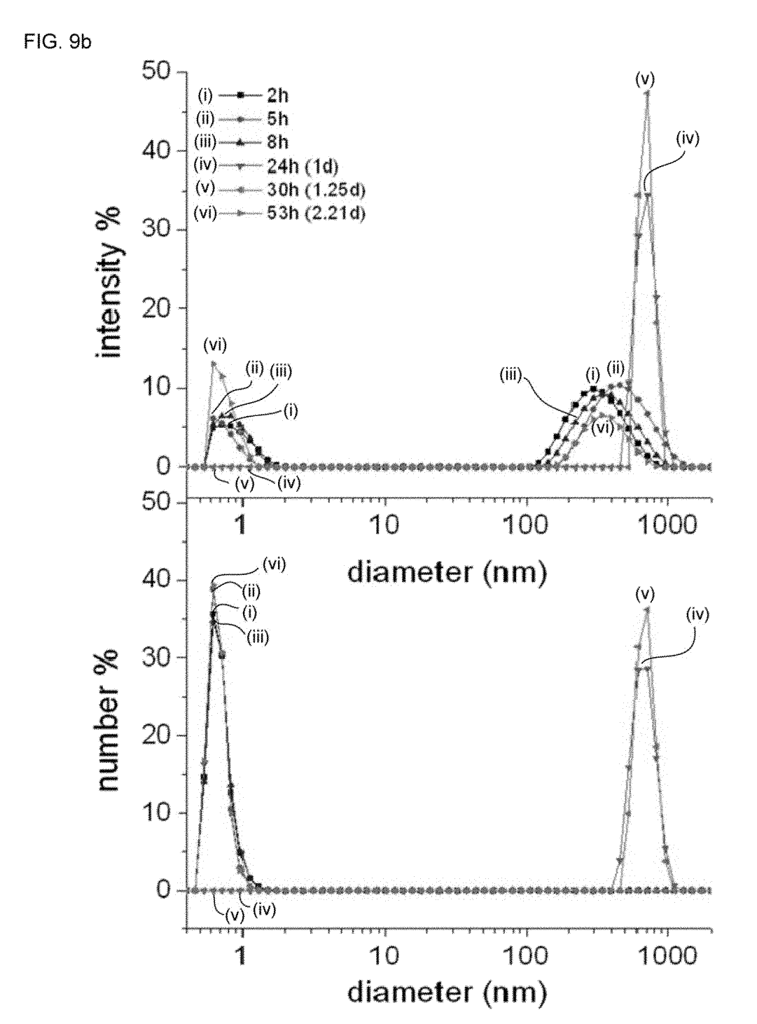

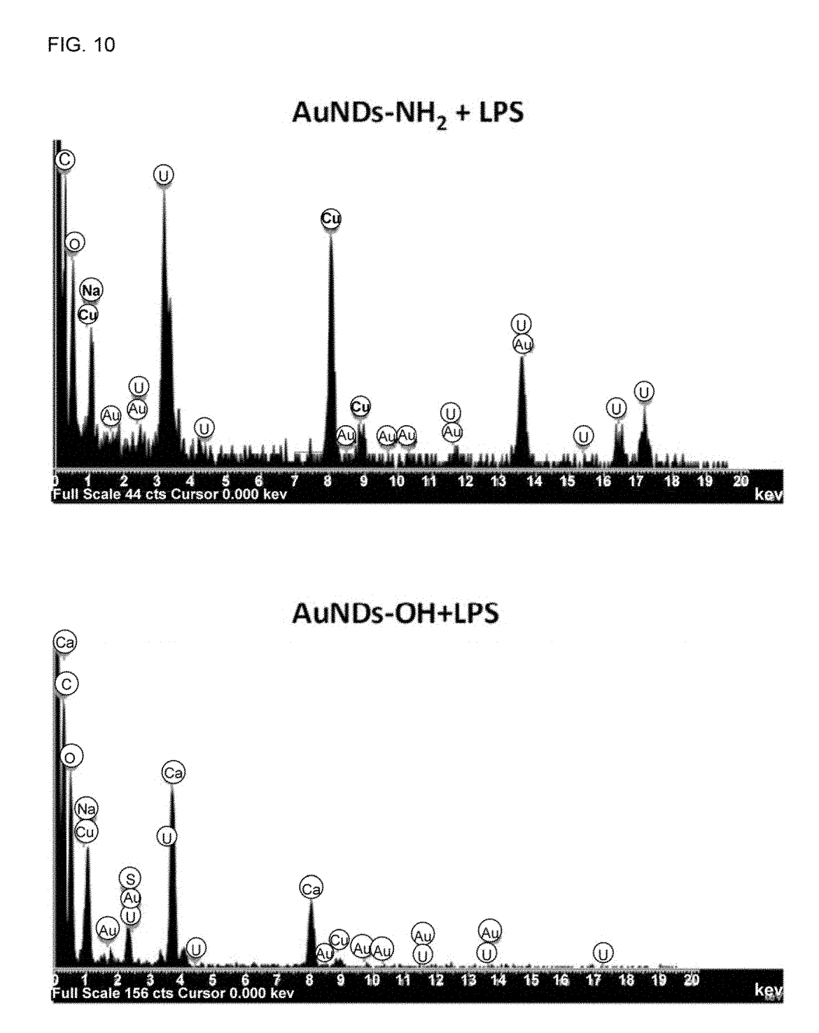

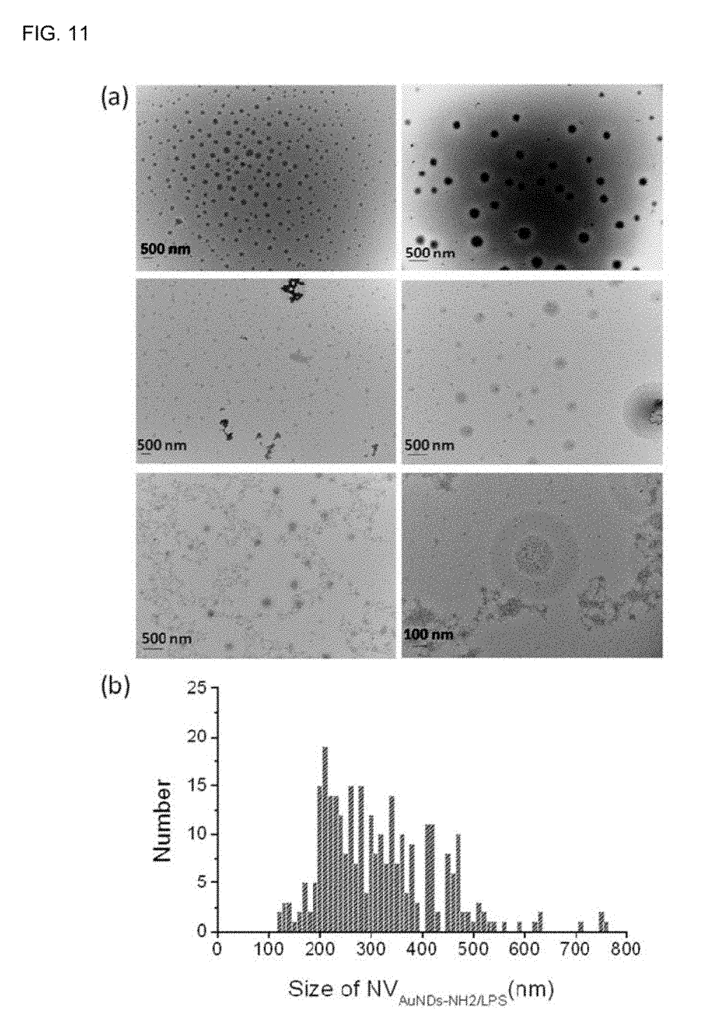

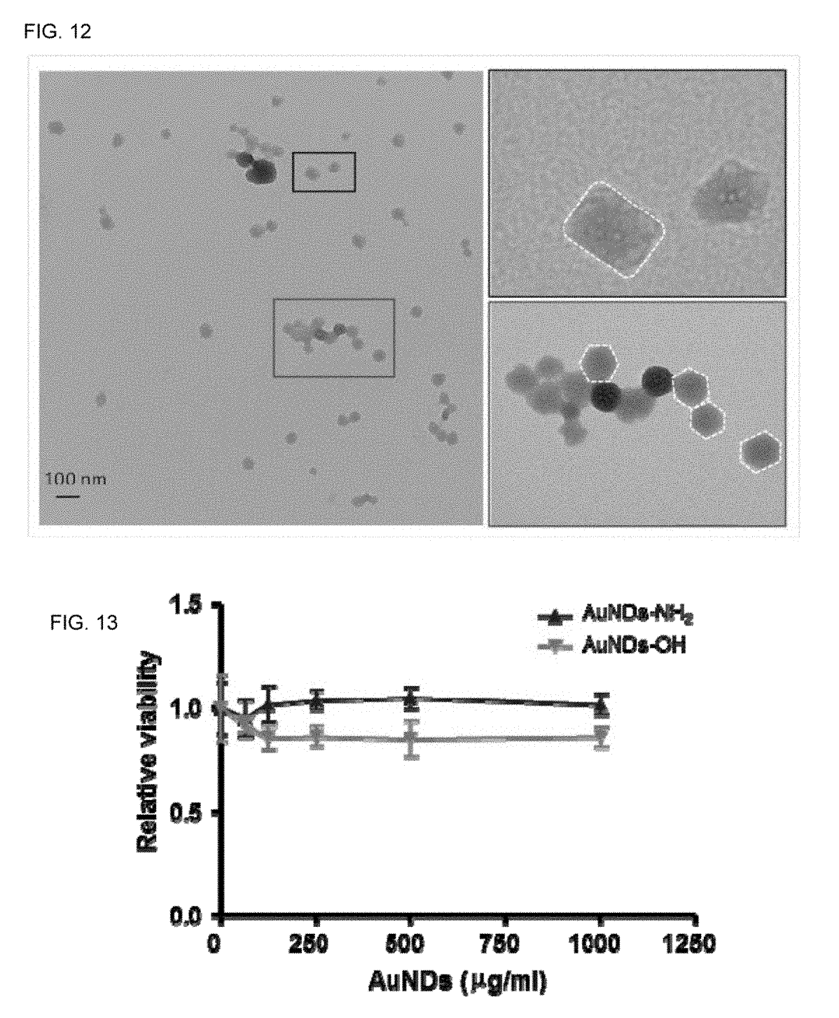

After mixing the LPS (i.e., endotoxin) with either AuNDs-OH (hydrophobicity) or AuNDs-NH₂ (hydrophilicity), a dramatic size increase can be found by dynamic light scattering (DLS) measurements (FIG. 9a). Despite the LPS concentration at 1 μg/mL (˜67 nM) is actually higher than a CMC value (˜41 nM)³⁷ at room temperature, the spontaneously self-assembled and disassembled processes are still being observed (FIG. 9b). This result indicated that the aggregation process of LPS-alone is dynamic. Comparatively, the LPS in the presence of AuNDs is found to be form more stable aggregates than LPS alone (see FIG. 9a). To further elucidate the detailed aggregation structures of LPS, we utilized transmission electron microscopy (TEM) to show that the spherical morphologies of AuNDs-OH/LPS aggregates (FIG. 2a, left panel) and AuNDs-NH₂/LPS aggregates (FIG. 2a, right panel) are very similar. However, the sizes (>200 nm) of the AuNDs-NH₂/LPS aggregates were almost all larger than those of the AuNDs-OH/LPS aggregates (142.8±22.8 nm). Interestingly, all the aggregates possessed an outer wall and an inner core with equivalent wall thicknesses. The thickness values of the AuNDs-OH/LPS and AuNDs-NH₂/LPS aggregations were 36.4±6.1 nm (FIG. 2b, gray bar) and 67.0±8.9 nm (FIG. 2b, black bar), respectively, which values exactly correspond to the length of a single LPS molecule and two LPS molecules, respectively. These results indicate that the LPS molecules adopt a tail-to-tail arrangement (with the lipid A portions as the tail domains) within the AuNDs-NH₂/LPS aggregates to form vesicle structures (hereafter denoted as NV_{AuNDs-NH2/LPS}). In contrast, the thinner walls of the AuNDs-OH/LPS aggregates indicated that these aggregates do not form vesicles. Because the average size (142.8±22.8 nm) of these aggregates was larger than that of regular micelles, this type of AuNDs-OH/LPS aggregate might be a form of large compound micelle (hereafter denoted as LCM_{AuNDs-OH/LPS}), in which the inside core is possibly filled with reverse micelle.^{16, 19} It can be deduced that such differences between the AuNDs-NH₂/LPS and AuNDs-OH/LPS aggregates might result from their different surface polarities, which might affect the LPS assembly. The hydrophilic AuNDs (i.e., AuNDs-NH₂) might provide a lateral force to link the polar domains of LPS molecules during their assembly. Subsequently, the lipid A assembly can be progressively extended until NV are formed. We speculated that the AuNDs-NH₂ can easily interact with the amine groups of the polar domains of LPS through a specific interaction. In order to further investigate this possibility, the amine groups of LPS were modified by methyl iodide to form quaternary ammonium ions (4°-ammonium ions). As expected, none of the various kinds of NV_{AuNDs-NH2/LPS} could be observed by TEM (data not shown), indicating that a specific interaction between the AuNDs and LPS molecules was eliminated after the chemical modification. It is still a challenge to observe the locations of AuNDs in NV_{AuNDs-NH2/LPS} and LCM_{AuNDs-OH/LPS} via high resolution TEM. This challenge, which results from their limited dimensions, has been mentioned in previous studies.^{19, 22} Instead, the presence of Au elements can be verified using energy-dispersive X-ray spectroscopy (FIG. 10). Additionally, TEM with dark field image (FIG. 2d) results showed that several vesicle surfaces presented a bright contrast, strongly suggesting that the AuNDs-NH₂ localized in the wall. As shown in FIG. 21, a further examination of the NV stability indicated that the sizes of the LPS aggregates were gradually increased through the incorporation of AuNDs-NH₂. Thus, it is essential to further clarify whether these NV can be transformed to Q or H after prolonging the incubation time. FIG. 2e shows that these fluid-like NV can fuse with each other, but cannot form the highly active Q and H. Such the fusion process would be random, a border rang from NV_{AuNDs-NH2/LPS} diameters can be predicted. After counting several TEM images (see FIG. 11a) with hundreds of NV_{AuNDs-NH2/LPS}, the size distribution is very large ranging from 120 nm to 800 nm (see FIG. 11b). Otherwise, another control set from LPS-alone (i.e., without the presence of AuNDs-NH₂) illustrated that the Q and H can indeed be observed (see FIG. 12) while being higher than the CMC, although their quantity is very limited. As a result, we were able to successfully suppress the transformation of NV_{AuNDs-NH2/LPS} and LCM_{AuNDs-OH/LPS} into Q and H through their association with AuNDs during LPS-assembly. About the pathway of two kinds of AuNDs in coassembly with LPS, it was simplified illustrated in FIG. 6. Again, it is worth noting the well-established fact that the Q and H of LPS exert overwhelming immunological responses. As such, the formation of stable NV_{AuNDs-NH2/LPS} would be conducive to allowing the naturally inflammatory activator (i.e., LPS) to act, at least in part, as a potential vaccine adjuvant for inducing specific immunological response.

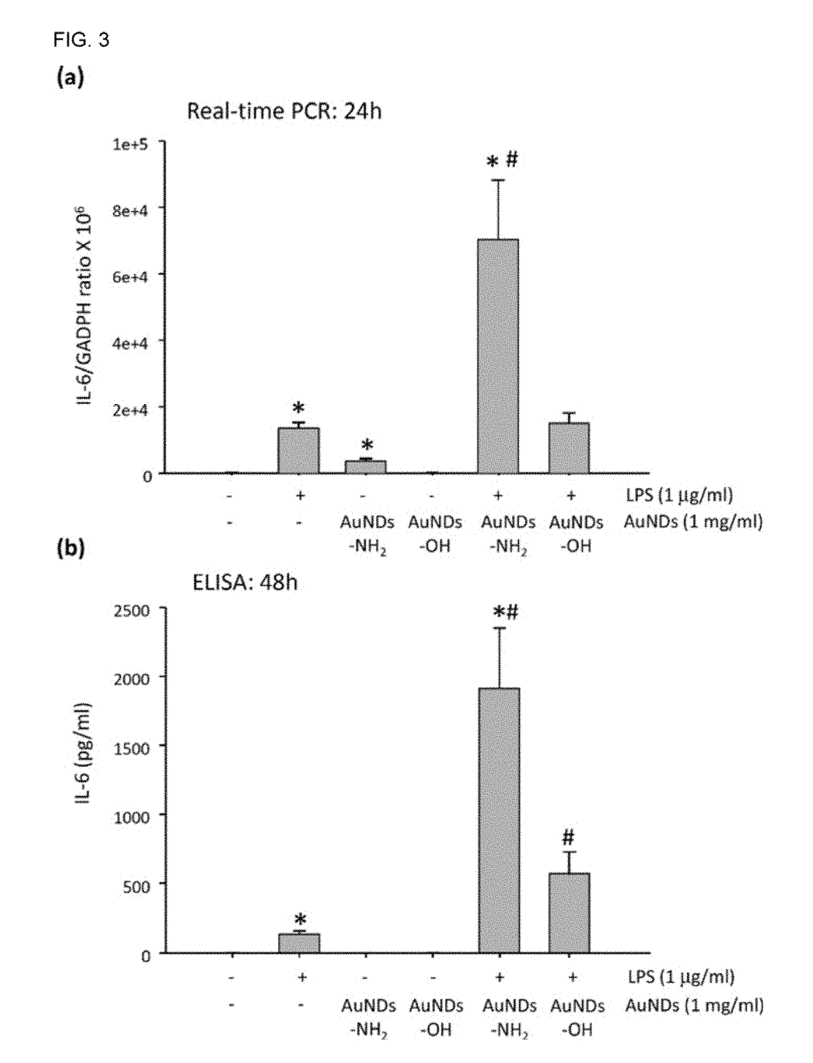

It is well known that vaccine adjuvants can trigger early innate inflammatory responses and initiate T-helper 1 (Th1) or T-helper 2 (Th2) responses.³⁸ Commonly used adjuvants usually initiate strong Th2 responses, but are rather ineffective against intracellular pathogens which require Th I-mediated immunity. Therefore, one of the challenges for vaccine adjuvant development is to select appropriate adjuvants which can effectively and selectively initiate Th1 or Th2 responses. Here, we intended to examine whether the NV_{AuNDs-NH2/LPS} and LCM_{AuNDs-OH/LPS} can effectively and specifically initiate Th1 or Th2 responses in human THP-1 cells. THP-1 cells are monocytic lineage cell lines which are differentiated into macrophage-like cells by treatment with phorbol 12-myristate 13-acetate (PMA). First, the immunological activity of NV_{AuNDs-NH2/LPS} and LCM_{AuNDs-OH/LPS} were determined by the production of proinflammatory cytokine interleukin-6 (IL-6). Both AuNDs have been examined and have been shown to be highly biocompatible (FIG. 13). The levels of interleukin-6 (IL-6) mRNA and protein production were determined using real-time reverse transcription-polymerase chain reaction (RT-PCR) and enzyme-linked immunosorbent assay (ELISA). As shown in FIGS. 3a and 3b, a greater degree of IL-6 induction can easily be observed from NV_{AuNDs-NH2/LPS} and LCM_{AuNDs-OH/LPS} in comparison to the amount of IL-6 induced by LPS-alone at 1 μg/ml, in which the lamellas or regular micelles still predominated within aggregates of LPS. It should be noted that since the AuNDs alone failed to trigger the IL-6 production in the THP-1 cells, we can rule out the possibility that AuNDs is either directly associated with TLRs or contaminated with immune stimulants.³⁹ Comparatively, the NV_{AuNDs-NH2/LPS} exhibited stronger proinflammatory IL-6 inducing capacity than the LCM_{AuNDs-OH/LPS} did. The difference can be attributed to the smaller size and lower lipid A density of LCM_{AuNDs-NH2/LPS} than NV_{AuNDs-NH2/LPS}, which in turn resulted in less effective induction of IL-6 production. Although not as dramatic. LCM_{AuNDs-OH/LPS} showed induction of IL-6 production as well. Our result showed that the aggregation structures of LCM_{AuNDs-OH/LPS} belong to non-typical micelles, which exhibit greater lipid A density than regular micelle (i.e. LPS-alone). Since lipid A is active component of LPS for immune system activation, the slightly higher lipid A density of LCM_{AuNDs-OH/LPS} may result in more effective induction of IL-6 production than LPS did.

We further investigated the innate chemotactic signals and inflammatory cytokines triggered by NV_{AuNDs-NH2/LPS} to characterize the features of adjuvant activity. By using a Luminex multiplex human cytokine ELISA, we found that the NV_{AuNDs-NH2/LPS} increased the productions of IL-6, IL-10, granulocyte colony-stimulating factor (G-CSF), interferon gamma-induced protein 10 (IP-10), and platelet-derived growth factor (PDGF)-BB; however, the NV_{AuNDs-NH2/LPS} decreased the productions of IL-7, IL-9, IL-12 (P70), basic fibroblast growth factor (FGF), and vascular endothelial growth factor (VEGF) compared with the production levels in the LPS-only group (see Table 1). LCM_{AuNDs-OH/LPS} may induce less obvious inflammatory signals and cytokines than NV_{AuNDs-NH2/LPS} do (Table 3). As a result of greater IL-6 induction, NV_{AuNDs-NH2/LPS} were chosen to determine the innate chemotactic signals and inflammatory cytokines. Table 1 shows cytokine and chemokine Levels in AuNDs-NH₂, LPS, and NV_{AuNDs-NH2/LPS}.

TABLE 1

| Cytokines | | | | |
|---|---|---|---|---|
| (pg/ml) | ddH₂O | AuNDs-NH₂ | LPS | NV_{AuNDs-NH2/LPS} |
| IL-1ra | 102.3 ± 13.3 | 111.7 ± 10.7 | 266.0 ± 5.3*** | 262.5 ± 35.8** |
| IL-2 | 8.3 ± 0.8 | 10.0 ± 0.46* | 12.2 ± 0.5** | 12.1 ± 1.1** |
| IL-4 | 1.5 ± 0.0 | 1.9 ± 0.2* | 3.4 ± 0.1*** | 3.4 ± 0.3*** |
| IL-6 | 4.8 ± 0.2 | 7.3 ± 1.6 | 45.3 ± 3.0*** | 122.0 ± 34.0**^{,#} |
| IL-7 | 4.3 ± 0.2 | 2.1 ± 0.3*** | 7.8 ± 0.5*** | 2.6 ± 0.2***^{,###} |
| IL-9 | 2.2 ± 0.2 | 3.2 ± 0.3** | 5.4 ± 0.5*** | 4.3 ± 0.2***^{,#} |
| IL-10 | 27.4 ± 6.0 | 37.5 ± 4.2 | 55.0 ± 1.5** | 86.6 ± 7.1***^{,##} |
| IL-12 (P70) | 116.8 ± 17.2 | 124.5 ± 5.1 | 174.6 ± 8.3** | 130.4 ± 23.5^{#} |
| IL-13 | 13.3 ± 1.5 | 13.3 ± 1.8 | 16.9 ± 1.0* | 15.0 ± 1.0 |
| IL-15 | 8.5 ± 1.0 | 13.6 ± 0.1*** | 15.7 ± 0.5*** | 16.8 ± 1.2*** |
| IL-17A | 13.0 ± 0.4 | 16.3 ± 1.9* | 20.4 ± 0.2*** | 21.1 ± 1.3*** |
| Basic FGF | 7.9 ± 0.1 | 8.7 ± 1.1 | 14.8 ± 1.0*** | 11.3 ± 0.3***^{,##} |
| CCL11 (Eotaxin) | 8.1 ± 1.1 | 10.2 ± 1.4 | 16.7 ± 0.8*** | 15.4 ± 0.4*** |
| G-CSF | 138.0 ± 0.5 | 206.3 ± 12.8*** | 423 ± 49.8*** | 1277.8 ± 243.3**^{,#} |
| GM-CSF | 254.3 ± 6.4 | 306.4 ± 8.3** | 368.5 ± 32.3** | 401.7 ± 8.3*** |
| IFN-r | 34.6 ± 3.1 | 42.4 ± 3.6** | 83.6 ± 4.6** | 75.9 ± 5.8*** |
| CXCL10 (IP-10) | 571.4 ± 179.2 | 1910.9 ± 990.1 | 1924.3 ± 809* | 3971.5 ± 631.6***^{,#} |
| CCL12 (MCP-1) | 50.6 ± 5.7 | 92.4 ± 42.7 | 667.7 ± 115.4*** | 367.9 ± 181.6* |
| PDGF-BB | 21.7 ± 1.8 | 27.0 ± 4.2 | 50.8 ± 3.3*** | 77.4 ± 6.9***^{,##} |
| TNF-α | 133.6 ± 15.2 | 296.7 ± 54.0** | 25183.1 ± 3739.5*** | 24471.8 ± 3129.0*** |
| VEGF | 907.3 ± 246.7 | 1199.8 ± 274.4 | 2842.2 ± 164.9*** | 1534.8 ± 581^{#} |

^{#}compared with LPS group. (^{#}P < 0.05; ^{##}P < 0.01; ^{###}P < 0.001)
*compared with ddH₂O group. (*P < 0.05; **P < 0.01; ***P < 0.001)

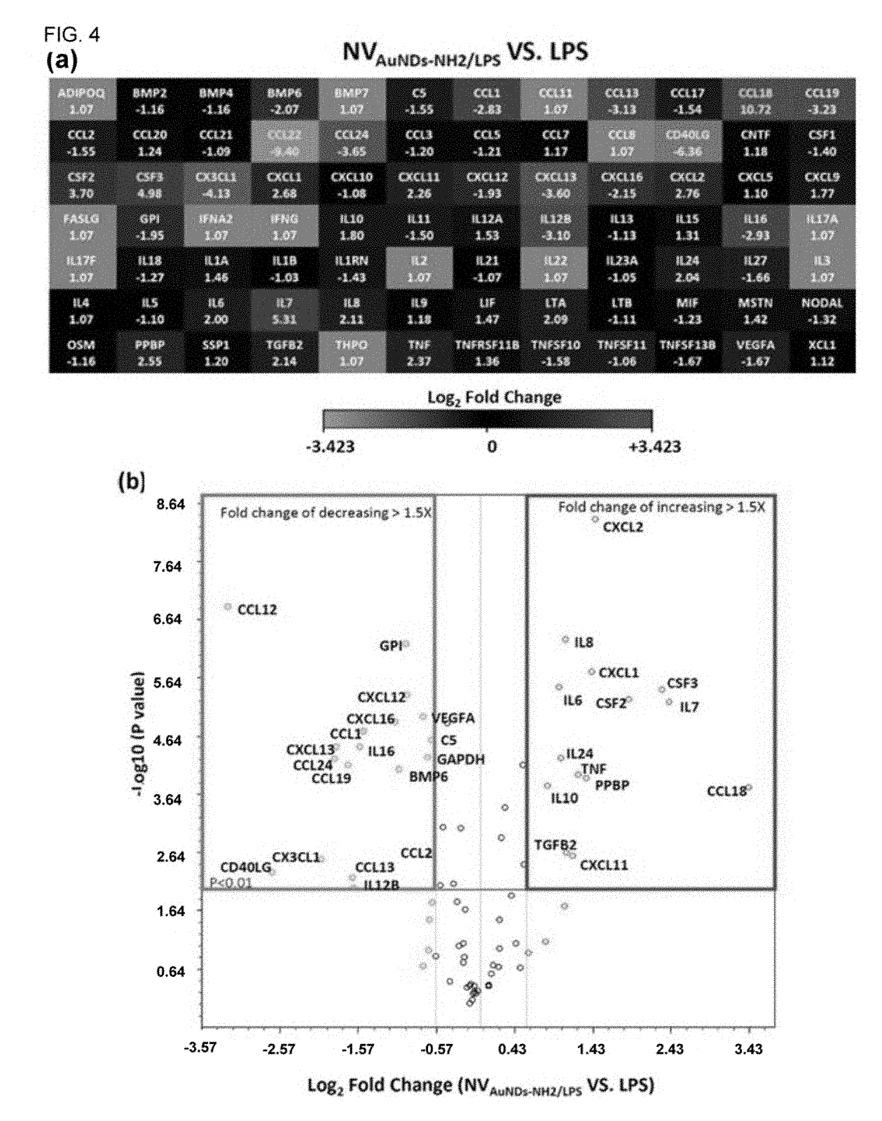

We used a cytokines and chemokines PCR array to determine the wide range of inflammatory mediators which were induced by NV_{AuNDs-NH2/LPS}. The PCR array results showed that, compared with LPS alone, the NV_{AuNDs-NH2/LPS} can modulate the mRNA levels of several cytokines and chemokines. FIG. 4a provides a graphical representation of fold regulation expression data between LPS and NV_{AuNDs-NH2/LPS} overlaid onto the PCR array plate layout. The increased cytokines triggered by endotoxin NV_{AuNDs-NH2/LPS} were IL-10, IL-24, IL-6, IL-7, IL-8, transforming growth factor-beta 2 (TGFB2), tumor necrosis factor (TNF), and lymphotoxin alpha (LTA); the upregulated chemokines were CCL18, CXCL1, CXCL2, and proplatelet basic protein (PPBP); and the upregulated growth factors were colony-stimulating factor 2 (CSF2) and colony-stimulating factor 3 (CSF3). The decreased cytokines were IL-12b, IL-16, and the TNF super-family CD40LG; the downregulated chemokines were CCL1, CCL3, CCL19, CCL2, CCL22, CCL24, CX3CL1, CXCL2. CXCL13, and CXCL16; and the downregulated growth factors were bone morphogenetic protein 6 (BMP6), and vascular endothelial growth factor A (VEGFA). The complement component 5 (C5) was also decreased (FIG. 4b, Table 4). Most of our cytokine ELISA result are correlated with gene expression, such as IL-6, IL-10, G-CSF (CSF-3), and VEGFA. However, IL-7 gene expressions are inconsistent with ELISA data. It has been reported that the half life of recombinant human IL-7 was pretty short in vivo.⁴⁰ It is possible that elevated IL-7 mRNA levels were still not high enough to reflect in IL-7 protein levels.

We further analyzed which pathways were triggered by NV_{AuNDs-NH2/LPS}. Table 5 displays the pathways that are statistically enriched among the upregulated and downregulated genes by using the comparative toxicogenomics database (CTD) analyzer. Most of the upregulated chemokines belonged to the CXC subfamily (CXCL1, CXCL2, CXCL7, IL-8, CXCL9, and CXCL11), and one belonged to the CC subfamily (CCL18). Most of the downregulated chemokines belonged to the CC subfamily (CCL1, CCL2, CCL13, CCL19, CCL22, and CCL24), one belonged to the CX3C subfamily (CX3CL1), and three belonged to the CXC subfamily (CXCL12, CXCL13, and CXCL6). The CTD analyzer provides more systematic and comprehensive information and allows for evaluations providing a complete view of NV_{AuNDs-NH2/LPS}-regulated pathways.

Finally, we also examined whether the NV_{AuNDs-NH2/LPS} influences THP-1 macrophage activations according to the PCR array results. Macrophages can response efficiently to environment stimulations and express differential cytokines and chemokines production to activate T helper type 1 (Th1) and Th2 cells. Table 2 showed that NV_{AuNDs-NH2/LPS} increase six Th1-associated cytokines and chemokines genes (e.g., IL-12A, TNFα, IL6, IL1α, CXCL-1, CXCL11) and three Th2-associated genes (e.g., IL-10. TCGFβ, CCL18). However, NV_{AuNDs-NH2/LPS} decrease three Th1-associated gene expressions (e.g., IL-12B, CXCl16, and CCL2) and six Th2-associated genes (e.g., VEGFA, CCL1, CCL13, CCL17, CCL22, CCL24). The increasing cytokines and chemokines conform to the majority of gene-expression profiles of Th1 adjuvant.³⁸ Table 2 shows differentially cytokines and chemokines gene expressions.

TABLE 2

| | Folds of gene expression | | |
|---|---|---|---|
| | compared with | | |
| Cytokines and | control group | | Effects of NV_{AuNDs-NH2/LPS} |
| chemokines | LPS | NV_{AuNDs-NH2/LPS} | treatment (>1.5X, P < 0.05) |
| IL12A | 1.43 | 2.18 | ↑ |
| IL12B | 11.79 | 3.81 | ↓ |
| TNFα | 6.76 | 16.01 | ↑ |
| IL6 | 40.52 | 81.16 | ↑ |
| IL1α | 21.64 | 31.50 | ↑ |
| IL23A | 22.21 | 21.21 | |
| CXCL1 | 9.41 | 25.17 | ↑ |
| CXCL2 | 7.96 | 21.94 | ↑ |
| CXCL9 | −1.54 | 1.15 | ↑ |
| CXCL10 | 4.65 | 4.30 | |
| CXCL11 | 2.65 | 5.99 | ↑ |
| CXCL16 | 1.65 | −1.30 | ↓ |
| CCL2 | 13.85 | 8.96 | ↓ |
| CCL3 | 11.09 | 9.25 | |
| CCL5 | 3.01 | 2.48 | |
| IL10 | 3.71 | 6.67 | ↑ |
| TGFB2 | −1.77 | 1.21 | ↑ |
| IL1RN | 3.22 | 2.25 | |
| VEGFA | 1.47 | −1.14 | ↓ |
| CCL1 | 12.27 | 4.34 | ↓ |
| CCL13 | 6.82 | 2.18 | ↓ |
| CCL17 | 3.63 | 2.36 | ↓ |
| CCL18 | −1.64 | 6.53 | ↑ |
| CCL20 | 4.02 | 4.97 | |
| CCL22 | 8.09 | −1.16 | ↓ |
| CCL24 | 7.47 | 2.05 | ↓ |

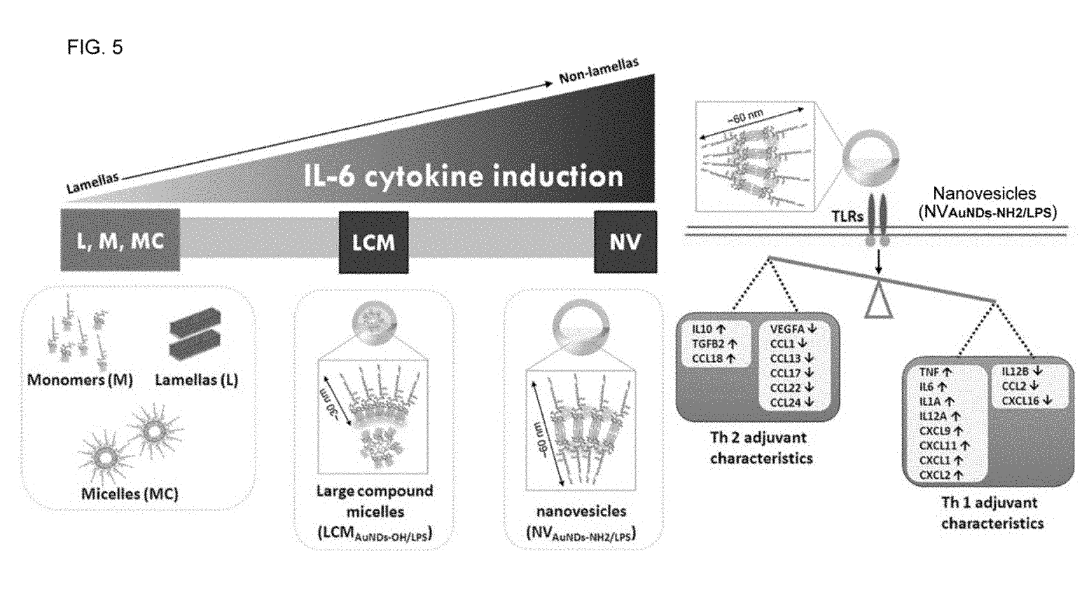

The CCL24, CCL17, and CCL22 are reported to recruit eosinophils, basophils, and Th2 cells, leading to a Th2 response.^{41, 42} CCL1 can promote the infiltration of eosinophils, Th2, and regulatory T cells.⁴³ IL-10 plays a critical role in limiting the duration and intensity of immune and inflammatory reactions. In macrophages, IL-10 inhibits the production of proinflammatory cytokines, such as TNF-α, IL-6, and IL-12, and downregulates the levels of MHC II and co-stimulatory molecules.⁴⁴ Moreover, IL-10 production is also believed as a feedback mechanism in response for TLR signaling.⁴⁵ TGFB is a pleiotropic cytokine that mediates a wide variety of effects on cellular differentiation, activation, and proliferation. It acts as a negative regulator to inhibit the LPS-induced macrophage production of the proinflammatory cytokines TNF-α, IL-1α, and IL-18. The NV_{AuNDs-NH2/LPS} increased IL-10 and TGFB2 productions might represent a macrophage feedback mechanism. Taken together, our results showed that the NV_{AuNDs-NH2/LPS} not only can efficiently elicit inflammatory cytokines and chemokine expressions, but also possess characteristic of T-helper 1 adjuvants; the above description is summarized in FIG. 5.

Table 3 shows cytokine and chemokine Levels in AuNDs-OH, LPS, and LCM_{AuNDs-OH/LPS}

TABLE 3

| Cytokines (pg/ml) | ddH₂O | AuNDs-OH | LPS | LCM_{AuNDs-OH/LPS} |
|---|---|---|---|---|
| IL-1ra | 120.9 ± 18.7 | 152.9 ± 19.9 | 771.0 ± 83.2*** | 887.5 ± 100.8*** |
| IL-2 | 0.2 ± 0.1 | 0.4 ± 0.3 | 7.0 ± 0.4*** | 6.5 ± 0.4*** |
| IL-4 | 0.4 ± 0.2 | 0.5 ± 0.2 | 2.9 ± 0.2*** | 2.8 ± 0.1*** |
| IL-6 | 2.5 ± 0.7 | 1.6 ± 2.1 | 137.9 ± 23.8*** | 573.6 ± 165.0***^{,##} |
| IL-7 | 4.7 ± 0.6 | 5.6 ± 0.4 | 9.1 ± 0.7*** | 10.5 ± 1.2*** |
| IL-9 | 0.9 ± 0.1 | 1.1 ± 0.3 | 15.5 ± 2.9*** | 13.0 ± 1.2*** |
| IL-10 | 15.8 ± 2.0 | 20.7 ± 1.4** | 140.0 ± 9.2*** | 132.5 ± 10.8*** |
| IL-12 (P70) | 40.2 ± 2.5 | 56.4 ± 2.0*** | 173.6 ± 10.0*** | 179.4 ± 10.1*** |
| IL-13 | 3.2 ± 0.2 | 4.6 ± 0.3*** | 8.8 ± 0.5*** | 9.1 ± 0.4*** |
| IL-15 | 0.6 ± 0.4 | 1.2 ± 0.7 | 15.7 ± 1.0*** | 14.5 ± 1.2*** |
| IL-17A | 2.2 ± 1.6 | 3.4 ± 0.5 | 43.2 ± 1.4*** | 43.6 ± 2.7*** |
| Basic FGF | 43.3 ± 9.9 | 43.1 ± 13.6 | 35.4 ± 0.9 | 34.5 ± 1.7 |
| CCL11 (Eotaxin) | 1.5 ± 0.6 | 1.5 ± 0.5 | 13.0 ± 0.7*** | 12.7 ± 0.6*** |
| G-CSF | 46.8 ± 2.2 | 46.4 ± 1.8 | 170.0 ± 12.9*** | 162.1 ± 18.1*** |
| GM-CSF | 37.8 ± 10.9 | 45.1 ± 4.0 | 118.8 ± 4.0*** | 111.1 ± 3.6*** |
| IFN-r | Undetectable | 5.2 ± 4.1 | 112.4 ± 6.2 | 108.9 ± 2.7 |
| CXCL10 (IP-10) | 432.9 ± 73.1 | 1093.8 ± 115.2*** | 3439.3 ± 764.4*** | 4561.5 ± 1376.5*** |
| CCL12 (MCP-1) | 2.5 ± 1.8 | 3.2 ± 1.2 | 749.2 ± 67.0*** | 594.4 ± 57.3***^{,#} |
| PDGF-BB | 4.9 ± 0.7 | 5.4 ± 1.2 | 16.3 ± 1.3*** | 18.7 ± 1.5*** |
| TNF-α | 2.3 ± 0.5 | 1.9 ± 0.2 | 606.1 ± 89.1*** | 591.2 ± 61.3*** |
| VEGF | 116.3 ± 13.6 | 193.5 ± 12.0*** | 1151.2 ± 127.1*** | 1268.9 ± 113.0*** |

^{#}compared with LPS group. (^{#}P < 0.05; ^{##}P < 0.01; ^{###}P < 0.001)
*compared with ddH₂O group. (*P < 0.05; **P < 0.01; ***P < 0.001)

Table 4 shows genes over-expressed and under-expressed in NV_{AuNDs-NH2/LPS} compared with LPS group.

TABLE 4

| Gene Symbol | Fold Regulation | P-value |
|---|---|---|
| CCL18 | 10.7235 | 0.000172 |
| CSF2 | 3.7004 | 0.000005 |
| CSF3 | 4.985 | 0.000004 |
| CXCL1 | 2.6761 | 0.000002 |
| CXCL11 | 2.2551 | 0.002615 |
| CXCL2 | 2.7551 | 0 |
| IL10 | 1.7985 | 0.000161 |
| IL24 | 2.0368 | 0.000055 |
| IL6 | 2.003 | 0.000003 |
| IL7 | 5.3106 | 0.000006 |
| IL8 | 2.1082 | 0 |
| LTA | 2.0949 | 0.019201 |
| PPBP | 2.5488 | 0.00012 |
| TGFB2 | 2.1399 | 0.002245 |
| TNF | 2.3676 | 0.000106 |
| BMP6 | −2.0693 | 0.000085 |
| C5 | −1.55 | 0.000027 |
| CCL1 | −2.8265 | 0.000019 |
| CCL13 | −3.1257 | 0.006199 |
| CCL19 | −3.2344 | 0.000072 |
| CCL2 | −1.5465 | 0.002516 |
| CCL22 | −9.4031 | 0 |
| CCL24 | −3.6485 | 0.000057 |
| CD40LG | −6.3644 | 0.00499 |
| CX3CL1 | −4.129 | 0.003005 |
| CXCL12 | −1.9302 | 0.000004 |
| CXCL13 | −3.6028 | 0.000035 |
| CXCL16 | −2.1453 | 0.000013 |
| GPI | −1.9532 | 0.000001 |
| IL12B | −3.0955 | 0.009343 |
| IL16 | −2.9295 | 0.000035 |
| VEGFA | −1.6724 | 0.000011 |
| ACTB | −2.8366 | 0.000019 |
| GAPDH | −1.6106 | 0.000052 |

Table 5 shows pathways that are statistically enriched among up-regulated and under-regulated genes by Comparative Toxicogenomics Database (CTD) analyzer.

TABLE 5

| | | | | | |
|---|---|---|---|---|---|
| | | | Corrected P- | Annotated | Genome |
| Pathway | Pathway ID | P-value | value | Genes | Frequency |

Up-regulated genes

| | | | | | |
|---|---|---|---|---|---|
| Cytokine- | KEGG: 04060 | 2.72e−27 | 1.49e−25 | 13 | 277/36684 |
| cytokine | | | | | genes: 0.76% |
| receptor | | | | | |
| interaction | | | | | |
| Jak-STAT | KEGG: 04630 | 1.69e−11 | 9.31e−10 | 6 | 158/36684 |
| signaling | | | | | genes: 0.43% |
| pathway | | | | | |
| Hematopoietic | KEGG: 04640 | 1.48e−10 | 8.12e−9 | 5 | 89/36684 |
| cell lineage | | | | | genes: 0.24% |
| Chemokine | KEGG: 04062 | 7.57e−9 | 4.16e−7 | 5 | 194/36684 |
| signaling | | | | | genes: 0.53% |
| pathway | | | | | |
| NOD-like | KEGG: 04621 | 7.80e−9 | 4.29e−7 | 4 | 63/36684 |
| receptor | | | | | genes: 0.17% |
| signaling | | | | | |
| pathway | | | | | |
| Toll-like | KEGG: 04620 | 8.58e−6 | 4.72e−4 | 3 | 107/36684 |
| receptor | | | | | genes: 0.29% |
| signaling | | | | | |
| pathway | | | | | |
| T cell receptor | KEGG: 04660 | 9.32e−6 | 5.13e−4 | 3 | 110/36684 |
| signaling | | | | | genes: 0.30% |
| pathway | | | | | |

down-regulated genes

| | | | | | |
|---|---|---|---|---|---|
| Cytokine- | KEGG: 04060 | 1.58e−26 | 1.19e−24 | 14 | 277/36684 |
| cytokine | | | | | genes: 0.76% |
| receptor | | | | | |
| interaction | | | | | |
| Chemokine | KEGG: 04062 | 1.20e−18 | 9.00e−17 | 10 | 194/36684 |
| signaling | | | | | genes: 0.53% |
| pathway | | | | | |

In conclusion, we use a simple strategy based on the utilization of hydrophilic AuNDs to control the supramolecular LPS assembly in order to facilitate the formation of stable endotoxin NV_{AuNDs-NH2/LPS}, thus avoiding the formation of highly active Q and H. In this way, the endotoxin NV_{AuNDs-NH2/LPS} can selectively exhibit T-helper 1 adjuvant activity, including the activity of IL-6 cytokine and several Th1-associated cytokines/chemokines. In comparison, the LCM_{AuNDs-OH/LPS} derived from hydrophobic AuNDs were less effective in terms of inflammatory cytokines production due to being smaller in size and having a lower lipid A density than the endotoxin NV_{AuNDs-NH2/LPS}. To the best of our knowledge, our study is the first to report such manipulation of the surface hydrophilicity of AuNDs to control LPS assembly and thereby avoid the formation of highly active Q and H. By involving the hydrophilic AuNDs to control LPS-elicited responses, it may be possible to promote T-helper 1-mediated immunity for application in specific vaccine development.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### REFERENCES

(1) Aderem, A.; Ulevitch, R. J. Nature 2000, 406, 782-787.
(2) Bohannon, J. K.; Hernandez, A.; Enkhbaatar, P.; Adams, W. L.; Sherwood, E. R. Shock 2013, 40, 451-462.
(3) Needham, B. D.; Carroll. S. M.; Giles, D. K.; Georgiou, G.; Whiteley. M.; Trent, M. S. Proc. Natl. Acad. Sci. USA 2013, 110, 1464-1469.
(4) Calabrese, V.; Cighetti, R.; Peri, F. Mol. Immunol. 2015, 63, 153-161.
(5) Park, B. S.; Lee, J. O. Exp. Mol. Med 2013, 45, e66.
(6) Brandenburg, K.; Mayer, H.; Koch, M. H. J.; Weckesser, J.; Rietschel, E. T.; Seydel. U. Eur. J. Biochem. 1993, 218, 555-563.
(7) Gutsmann, T.; Schromm, A. B.; Brandenburg, K. Int. J. Med Microbiol. 2007, 297, 341-352.
(8) Sigalov, A. B. Mol. Immunol. 2012, 51, 356-362.
(9) Molinaro, A.; Hoist, O.; Di Lorenzo, F.; Callaghan, M.; Nurisso, A.; D'Errico G.; Zamyatina, A.; Peri, F.; Berisio, R.; Jerala, R.; Jimenez-Barbero. J.; Silipo, A.; Martin-Santamaria, S. Chem. Eur. J. 2015, 21, 500-519.
(10) Seydel, U.; Oikawa, M.; Fukase, K.; Kusumoto, S.; Brandenburg, K. Eur J. Biochem. 2000, 267, 3032-3039.
(11) Seydel, U.; Hawkins, L.; Schromm, A. B.; Heine, H.; Scheel, O.; Koch, M. H. J.; Brandenburg. K. Eur. J. Immunol. 2003, 33, 1586-1592.
(12) Brandenburg, K.; Hawkins, L.; Garidel, P.; Andra, J.; Muller, M.; Heine, H.; Koch, M. H. J.; Seydel, U. Biochemistry 2004, 43, 4039-4046.
(13) Pontes, F. J. S.; Rusu, V. H.; Soares, T. A.; Lins, R. D. J. Chem. Theory Comput. 2012, 8, 3830-3838.
(14) Adams, P. G.; Lamoureux, L.; Swingle, K. L.; Mukundan, H.; Montano, G. A. Biophys. J. 2014, 106, 2395-2407.
(15) Nascimento, A., Jr.; Pontes, F. J. S.; Lins, R. D.; Soares, T. A. Chem. Commun. 2014, 50, 231-233.
(16) Zhang, L. F.; Eisenberg. A. J. Am. Chem. Soc. 1996, 118, 3168-3181.
(17) Chiu, J. J.; Kim, B. J.; Kramer. E. J.; Pine, D. J. J. Am. Chem. Soc. 2005, 127, 5036-5037.
(18) Kim, B. S.; Qiu, J. M.; Wang, J. P.; Taton, T. A. Nano Lett. 2005, 5, 1987-1991.
(19) Sanchez-Gaytan, B. L.; Cui, W.; Kim, Y.; Mendez-Polanco, M. A.; Duncan, T. V.; Fryd. M.; Wayland, B. B.; Park, S.-J. Angew. Chem. Int. Ed. 2007, 46, 9235-9238.
(20) Chen, T.; Yang, M.; Wang. X.; Tan. L. H.; Chen, H. J. Am. Chem. Soc. 2008, 130, 11858-11859.
(21) Kamps, A. C.; Sanchez-Gaytan, B. L.; Hickey, R. J.; Clarke, N.; Fryd, M.; Park, S.-J. Langmuir 2010, 26, 14345-14350.
(22) Mai, Y.; Eisenberg, A. J. Am. Chem. Soc. 2010, 132, 10078-10084.
(23) Fuks, G.; Talom, R. M.; Gauffre, F. Chem. Soc. Rev. 2011, 40, 2475-2493.
(24) Mai, Y.; Eisenberg, A. Macromolecules 2011, 44, 3179-3183.
(25) Sanchez-Gaytan. B. L.; Li, S.; Kamps, A. C.; Hickey, R. J.; Clarke, N.; Fryd, M.; Wayland, B. B.; Park, S. J. J. Phys. Chem. C 2011, 115, 7836-7842.
(26) Hickey, R. J.; Meng, X.; Zhang, P.; Park, S.-J. ACS Nano 2013, 7, 5824-5833.
(27) Aota-Nakano, U.; Li, S. J.; Yamazaki, M. Biochim. Biophys. Acta Biomembranes 1999, 1461, 96-102.
(28) Li, S. J.; Yamashita, Y.; Yamazaki, M. Biophys. J. 2001, 81, 983-993.
(29) Masum, S. M.; Li, S. J.; Tamba, Y.; Yamashita, Y.; Tanaka, T.; Yamazaki, M. Langmuir 2003, 19, 4745-4753.
(30) Masahito, Y., Transformation between liposomes and cubic phases of biological lipid membranes induced by modulation of electrostatic interactions. In Advances in Planar Lipid Bilayers and Liposomes, Leitmannova Liu, A., Ed. Academic Press: San Diego, Calif., 2009; Vol. 9, pp 163-209.
(31) Muir, B. W.; Zhen, G. L.; Gunatillake, P.; Hartley, P. G. J Phys. Chem. B 2012, 116, 3551-3556.
(32) Xu, J.; Han. Y.; Cui, J.; Jiang, W. Lanmgmuir 2013, 29, 10383-10392.
(33) Chien, C. T.; Liu, C. Y.; Wu, Z. W.; Chen, P. J.; Chu, C. L.; Lin, S. Y. J. Mater. Chem. B 2014, 2, 6730-6737.
(34) Ratoi, M.; Hoet, P. H. M.; Crossley, A.; Dobson, P. RSC Adv. 2014, 4, 20573-20581.
(35) Glushko, V.; Thaler, M. S. R.; Karp, C. D. Arch. Biochem. Biophys. 1981, 210, 33-42.
(36) Chen, W.; Tomalia, D. A.; Thomas, J. L. Macromolecules 2000, 33, 9169-9172.
(37) Sasaki, H.; White, S. H. Biophys. J. 2008, 95, 986-993.
(38) Korsholm, K. S.; Petersen, R. V.; Agger, E. M.; Andersen, P. Immunology 2010, 129, 75-86.
(39) Vallhov, H.; Qin, J.; Johansson, S. M.; Ahlborg, N.; Muhammed, M. A.; Scheynius, A.; Gabrielsson, S. Nano Lett. 2006, 6, 1682-1686.
(40) Sportes, C.; Babb, R. R.; Krumlauf, M. C.; Hakim, F. T.; Steinberg, S. M.; Chow, C. K.; Brown, M. R.; Fleisher, T. A.; Noel, P.; Maric, I.; Stetler-Stevenson, M.; Engel, J.; Buffet, R.; Morre, M.; Amato, R. J.; Pecora, A.; Mackall, C. L.; Gress, R. E. Clin. Cancer Res. 2010, 16, 727-735.
(41) Mantovani, A.; Sozzani, S.; Locati, M.; Allavena, P.; Sica, A. Trends Immunol. 2002, 23, 549-555.
(42) Martinez. F. O.; Gordon, S.; Locati, M.; Mantovani, A. J. Immunol. 2006, 177, 7303-7311.
(43) Hao, N. B.; Lu, M. H.; Fan, Y. H.; Cao, Y. L.; Zhang, Z. R.; Yang, S. M. Clin. Dev. Immunol. 2012.
(44) Martinez, F. O.; Sica, A.; Mantovani, A.; Locati, M. Front Biosci. 2008, 13, 453-461.
(45) Iyer, S. S.; Ghaffari, A. A.; Cheng, G. J. Immunol. 2010, 185, 6599-6607.

1. An endotoxin aggregate, comprising:
(a) lipopolysaccharide molecules, assembled into a vesicle with a wall surrounding an inner space or a spherical aggregate with a wall surrounding an inner core; and
(b) hydrophilic gold nanodots or gold nanoparticles localized in the wall of the vesicle, or hydrophobic gold nanodots or gold nanoparticles localized in the wall of the of the spherical aggregate.

2. The endotoxin aggregate of claim 1, wherein the gold nanodots or gold nanoparticles are embedded within a dendrimer and form a gold nanodot- or a gold nanoparticle-dendrimer complex.

3. The endotoxin aggregate of claim 2, wherein the dendrimer has branched amines or branched hydroxyl groups.

4. The endotoxin aggregate of claim 2, wherein the dendrimer is a generation-4 dendrimer.

5. The endotoxin aggregate of claim 2, wherein the gold nanodot or gold nanoparticle-dendrimer complex exhibits a hydrophilic surface polarity.

6. An endotoxin nanovesicle, comprising:
(a) lipopolysaccharide molecules, assembled into a vesicle with a wall surrounding an inner space; and
(b) hydrophilic gold nanodots or gold nanoparticles, localized in the wall of the vesicle.

7. The nanovesicle of claim 6, wherein the hydrophilic gold nanodots or gold nanoparticles are confined inside of a dendrimer with branched amines.

8. The nanovesicle of claim 6, wherein the hydrophilic gold nanodots or gold nanoparticles interact with amine groups of the lipopolysaccharide molecules.

9. The nanovesicle of claim 6, wherein the gold nanodots or gold nanoparticles are not alkanethiol-stabilized.

10. The nanovesicle of claim 6, wherein the wall of the vesicle has a thickness of about the length of two lipopolysaccharide molecules.

11. The nanovesicle of claim 6, wherein the lipopolysaccharide molecules adopt a lipid A-tail-to-lipid A-tail arrangement.

12. The endotoxin aggregate of claim 1, which is free of cubosomes and/or hexosomes.

13. The endotoxin aggregate of claim 1, wherein the spherical aggregate is a large compound micelle with the inner core filled with reverse micelle.

14. A composition comprising:
(a) the endotoxin nanovesicle of claim 6; and
(b) optionally an immunogenic antigen.

15. A composition comprising:
(a) the endotoxin aggregate of claim 13; and
(b) optionally an immunogenic antigen.

16. A method of preparing a lipopolysaccharide adjuvant, comprising:
(a) admixing lipopolysaccharide molecules with hydrophilic gold nanodots or gold nanoparticles; and
(b) allowing the lipopolysaccharide molecules to aggregate and form the endotoxin nanovesicle of claim 5, and thereby preparing the lipopolysaccharide adjuvant.

17. A method of suppressing formation of cubosomes and/or hexosomes in lipopolysaccharide aggregation or assembly, comprising:
(a) admixing lipopolysaccharide molecules with hydrophilic or hydrophobic gold nanodots or gold nanoparticles; and
(b) allowing the lipopolysaccharide molecules to assemble and form the endotoxin aggregate of claim 1.

18. A method of suppressing formation of cubosomes and/or hexosomes in lipopolysaccharide aggregation or assembly, comprising:
(a) admixing lipopolysaccharide molecules with hydrophilic gold nanodots or gold nanoparticles; and
(b) allowing the lipopolysaccharide molecules to assemble and form the endotoxin nanovesicle of claim 6.

19. A method of enhancing type 1 T helper cell-induced immunological responses in a subject in need thereof, comprising:
administering to the subject in need thereof an effective amount of the composition of claim 14, and thereby enhancing the type 1 T helper cell-induced immunological responses in the subject in need thereof.

20. The endotoxin aggregate of claim 1, which is free of lipopolysaccharide-assembled micelles and/or lipopolysaccharide-assembled lamellas.
